(19) United States
(12) Patent Application Publication
(10) Pub. No.: US 2015/0209432 A1
(43) Pub. Date: Jul. 30, 2015

(54) PHARMACEUTICAL COMPOSITIONS OF PROTON PUMP INHIBITOR

(71) Applicant: LUPIN LIMITED, Mumbai, Maharashtra (IN)

(72) Inventors: Kishore Kumar Konda, Pune (IN); Pandarinath Jadhav, Pune (IN); Satish Kumar Dalal, Pune (IN); Shirish Kumar Kulkarni, Pune (IN)

(73) Assignee: LUPIN LIMITED, Mumbai, Maharashtra (IN)

(21) Appl. No.: 14/417,241

(22) PCT Filed: Jul. 22, 2013
(86) PCT No.: PCT/IB2013/056003

§ 371 Date: Jan. 26, 2015

(30) Foreign Application Priority Data
Jul. 26, 2012 (IN) 838/KOL/2012

(51) Int. Cl. A61K 47/32; A61K 47/02; A61K 9/50; A61K 31/4439

(52) U.S. Cl. CPC A61K 47/32; A61K 31/4439; A61K 47/02; A61K 9/5073

(57) ABSTRACT

The present invention relates to pharmaceutical compositions of Proton Pump Inhibitor and process for preparation of such composition thereof. The invention relates to pharmaceutical compositions comprising Proton Pump Inhibitor, crospovidone, basic inorganic salt and one or more pharmaceutically acceptable excipients. The present invention provides stable pharmaceutical compositions of Dexlansoprazole which are bioequivalent to marketed dual delayed release pharmaceutical compositions of Dexlansoprazole.

### FIELD OF THE INVENTION

The present invention relates to pharmaceutical compositions comprising Proton Pump Inhibitor (PPI) and a process for preparation thereof. Further, present invention provides stable pharmaceutical compositions of Proton Pump Inhibitor and process for preparation thereof.

### BACKGROUND OF THE INVENTION

A benzimidazole-based compound has a strong inhibitory action on the proton pump, and it is widely used as a therapeutic agent for stomach ulcer, duodenal ulcer etc., by inhibiting gastric acid secretion. These compounds are well known as “Proton Pump Inhibitors” (PPIs).

Proton Pump Inhibitors include but not limited to Lansoprazole, Omeprazole, Rabeprazole, Pantoprazole, Leminoprazole, Tenatoprazole, or an optically active isomer thereof, or a pharmaceutically acceptable salt thereof.

However, these compounds are inferior in stability, and are unstable to humidity, temperature and light. In particular, they are unstable to acids and, when formulated into an aqueous solution or a suspension, they become extremely unstable with lowering of pH.

There are various techniques developed to stabilize PPIs.

U.S. Pat. No. 8,105,626 discloses capsule composition of lansoprazole-containing granules, comprising a principal ingredient layer comprising lansoprazole or an optically active isomer thereof or a pharmaceutically acceptable salt thereof in an amount of 12% by weight or more based on the total granules, and one or more basic inorganic salts in an amount of 0.2 part by weight to 0.4 part by weight based on 1 part by weight of lansoprazole or the optically active isomer thereof or the pharmaceutically acceptable salt thereof.

U.S. Pat. No. 7,220,762 discloses a method for stabilizing a benzimidazole-based compound comprising incorporating by mixing together (1) a crospovidone, (2) sodium hydroxide, potassium hydroxide or a mixture thereof, and (3) a benzimidazole-based compound thereby forming a uniform mixture of components (1), (2) and (3); wherein the benzimidazole-based compound (3) and the crospovidone (1) are incorporated with each other in a weight ratio of 0.5 to 5 part by weight to 1 part by weight of the benzimidazole-based compound and, wherein the sodium hydroxide or potassium hydroxide or their mixture (2) and the benzimidazole-based compound (3) are incorporated with each other in a weight ratio of 0.01 to 2 parts (2) to one part by weight of the benzimidazole-based compound (3) and, wherein the crospovidone (1) is present in an amount of more than 10 weight percent of the mixture.

While there are many compositions available to improve stability of PPI compositions, still there remains a need to develop a stable pharmaceutical composition of PPI.

### OBJECTIVE OF THE INVENTION

The main object of invention is to provide pharmaceutical compositions of Proton Pump Inhibitor.

Another object of invention is a pharmaceutical composition comprising a proton pump inhibitor, crospovidone, basic inorganic salt and one or more pharmaceutically acceptable excipient, wherein the crospovidone is present in an amount less than 0.5 part by weight based on one part by weight of the proton pump inhibitor.

Another object of invention is a pharmaceutical composition comprising a proton pump inhibitor, crospovidone, basic inorganic salt and one or more pharmaceutically acceptable excipient, wherein the crospovidone is present in an amount less than 0.5 part by weight based on one part by weight of the proton pump inhibitor, wherein the basic inorganic salt is present in an amount less than 0.2 part by weight based on one part by weight of proton pump inhibitor.

Another object of invention is a pharmaceutical composition comprising a Dexlansoprazole, crospovidone, basic inorganic salt and one or more pharmaceutically acceptable excipient, wherein the crospovidone is present in an amount less than 0.5 part by weight based on one part by weight of Dexlansoprazole, wherein the basic inorganic salt is present in an amount less than 0.2 part by weight based on one part by weight of Dexlansoprazole, wherein Cₘₐₓ and AUC of the composition are within the limit of 80% to 125% of Cₘₐₓ and AUC of the dual delayed capsule formulation composition of Dexlansoprazole.

Another object of invention is a pharmaceutical composition comprises granules, wherein the granules comprising an active layer comprising proton pump inhibitor, Crospovidone and basic inorganic salt; an intermediate coating layer formed on active layer; and an enteric coating layer formed on the intermediate coating layer, wherein the basic inorganic salt is present in an amount less than 0.2 part by weight based on one part by weight of proton pump inhibitor.

Another object of invention is a method of treating heartburn, acid reflux or gastroesophageal reflux disease in a patient, comprising administering pharmceutical composition comprises granules, wherein the granules comprising an active layer comprising Dexlansoprazole, Crospovidone and basic inorganic salt; an intermediate coating layer formed on active layer; and an enteric coating layer formed on the intermediate coating layer, wherein the basic inorganic salt is present in an amount less than 0.2 part by weight based on one part by weight of Dexlansoprazole.

### SUMMARY OF THE INVENTION

The present invention relates to pharmaceutical compositions of Proton Pump Inhibitor. The invention relates to pharmaceutical compositions comprising Proton Pump Inhibitor, crospovidone, basic inorganic salt and one or more pharmaceutically acceptable excipients. The present invention provides stable pharmaceutical compositions of Proton pump inhibitor for a period of three months when stored under conditions of 40° C./75% RH (Relative humidity).

### DETAILED DESCRIPTION OF THE INVENTION

The present invention relates to pharmaceutical compositions of Proton Pump Inhibitor, more particularly, the invention relates to pharmaceutical compositions of Proton Pump Inhibitor which are stable and a process for preparation thereof.

The term, “Proton Pump Inhibitor” (PPI) includes but not limited to Lansoprazole, Omeprazole, Rabeprazole, Pantoprazole, Leminoprazole, Tenatoprazole.

It will be understood that, for the purposes of the invention, PPI will include all forms of these compounds and not limited to their base, pharmaceutically acceptable salt(s) like Omeprazole magnesium, Esomeprazole sodium or optically active isomer(s) like Esomeprazole, Dexlansoprazole or polymorph(s) thereof.

Proton Pump Inhibitors used in pharmaceutical compositions of invention in an amount of which is safe, well tolerated in patients with acceptable adverse effect profiles and are those in common practice and known to person skilled in the art.

A pharmaceutical composition according to the invention comprises but is not limited to tablets (single layered tablets, multilayered tablets, mini tablets, bioadhesive tablets, caplets, matrix tablets, tablet within a tablet, mucoadhesive tablets, modified release tablets, pulsatile release tablets, and timed release tablets), pellets, beads, granules, sustained release formulations, capsules, microcapsules, tablets in capsules, microspheres, matrix formulations, microencapsulation.

In one embodiment, a pharmaceutical composition comprising a proton pump inhibitor, crospovidone, basic inorganic salt and one or more pharmaceutically acceptable excipient, wherein the crospovidone is present in an amount less than 0.5 part by weight based on one part by weight of the proton pump inhibitor.

The term “basic inorganic salt” includes but not limited to basic inorganic salts of sodium, potassium, magnesium and calcium.

Examples of the basic inorganic salt of sodium include sodium carbonate, sodium bicarbonate, sodium hydroxide and the like. Examples of the basic inorganic salt of potassium include potassium carbonate, potassium bicarbonate, potassium hydroxide and the like. Examples of the basic inorganic salt of magnesium include ground magnesium carbonate, magnesium carbonate, magnesium oxide, magnesium hydroxide, magnesium aluminate metasilicate, magnesium silicate, magnesium aluminate, synthetic hydrotalcite [Mg₆Al₂(OH)₁₆.CO₃.4H₂O] and alumina magnesium hydroxide [2.5MgO.Al₂O₃×H₂O], preferably, ground magnesium carbonate, magnesium carbonate, magnesium oxide, magnesium hydroxide and the like.

Examples of the basic inorganic salt of calcium include precipitated calcium carbonate, calcium hydroxide and the like. The basic inorganic salt used in the present invention may be a salt whose 1% aqueous solution or suspension has basic pH (pH 7 or higher). The basic inorganic salt may be formulated alone or in combination of two or more thereof, and the amount to be formulated is less than 0.2 by weight based on 1 part by weight of Proton Pump Inhibitor.

In another embodiment, a pharmaceutical composition comprising Dexlansoprazole, crospovidone, basic inorganic salt and one or more pharmaceutically acceptable excipient, wherein the crospovidone is present in an amount less than 0.5 part by weight based on one part by weight of the Dexlansoprazole.

In another embodiment, a pharmaceutical composition is capsule comprising a proton pump inhibitor, crospovidone, basic inorganic salt and one or more pharmaceutically acceptable excipient, wherein the crospovidone is present in an amount less than 0.5 part by weight based on one part by weight of the proton pump inhibitor.

In another embodiment, a pharmaceutical composition comprising a proton pump inhibitor, crospovidone, basic inorganic salt and one or more pharmaceutically acceptable excipient, wherein the crospovidone is present in an amount less than 0.5 part by weight based on one part by weight of the proton pump inhibitor, wherein the basic inorganic salt is present in an amount less than 0.2 part by weight based on one part by weight of proton pump inhibitor.

In another embodiment, a pharmaceutical composition is capsule comprising a proton pump inhibitor, crospovidone, basic inorganic salt and one or more pharmaceutically acceptable excipient, wherein the crospovidone is present in an amount less than 0.5 part by weight based on one part by weight of the proton pump inhibitor; wherein the basic inorganic salt is present in an amount less than 0.2 part by weight based on one part by weight of proton pump inhibitor.

In another embodiment, a pharmaceutical composition comprising a Dexlansoprazole, crospovidone, basic inorganic salt and one or more pharmaceutically acceptable excipient, wherein the crospovidone is present in an amount less than 0.5 part by weight based on one part by weight of Dexlansoprazole, wherein the basic inorganic salt is present in an amount less than 0.2 part by weight based on one part by weight of Dexlansoprazole.

In another embodiment, a pharmaceutical composition comprising a Dexlansoprazole, crospovidone, potassium hydroxide and one or more pharmaceutically acceptable excipient, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole.

In another embodiment, a pharmaceutical composition is capsule comprising a Dexlansoprazole, crospovidone, potassium hydroxide and one or more pharmaceutically acceptable excipient, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole.

In another embodiment, a pharmaceutical composition comprises granules, wherein the granules comprising an active layer comprising proton pump inhibitor, Crospovidone and basic inorganic salt; an intermediate coating layer formed on active layer; and an enteric coating layer which is formed on the intermediate coating layer, wherein the basic inorganic salt is present in an amount less than 0.2 part by weight based on one part by weight of proton pump inhibitor.

In another embodiment, active layer is formed by coating cores or inert pellets. Cores or inert pellets are composed of one or more materials selected from sucrose, starch, lactose and microcrystalline cellulose. Examples of the cores include a spherical granulated material of sucrose and starch, a spherical granulated material of cellulose, a spherical granulated material of cellulose and lactose, etc.

Cores or inert pellets are coated with the active layer comprising proton pump inhibitor, Crospovidone, basic inorganic salt and one more pharmaceutically acceptable excipients. It is desirable that cores are as uniformly spherical as possible so as to reduce variability of coating.

“Intermediate coating layer” is a coating layer which can prevent direct contact between Proton Pump Inhibitor and an enteric coating layer because the enteric coating layer component is an acidic substance. Intermediate coating layer comprises one or more pharmaceutically acceptable excipients like antistatic agents and masking agents.

For example, intermediate coating layer is a layer in which a saccharide such as sucrose, starch sugar such as corn starch, lactose, honey, sugar alcohol (D-mannitol, erythritol, etc.), etc. is appropriately formulated into a polymer base such as low-substituted hydroxypropylcellulose, hydroxypropylcellulose, hydroxypropylmethylcellulose (e.g. TC-5, etc.), polyvinylpyrrolidone, polyvinyl alcohol, methylcellulose, hydroxyethylmethylcellulose and the like. In addition, the intermediate coating layer may contain masking agent like titanium oxide and an antistatic agent like titanium oxide, talc, etc.

In another embodiment, a pharmaceutical composition of invention comprises one or more intermediate coating layers between active layer and enteric coating layer.

“Enteric coating layer” comprises one or more enteric polymers and one more pharmaceutically acceptable excipients comprise but not limited to sustained release agents like ethyl acrylate-methacrylic acid copolymer, ethyl cellulose.

Enteric coating layer comprise enteric polymers selected from the group comprising hydroxypropylmethyl cellulose phthalate, cellulose acetate phthalate, carboxymethylethyl cellulose, methyl methacrylate-methacrylic acid copolymer like Eudragit L 100, methacrylic acid-ethyl acrylate copolymer like Eudragit L 30 D55, methacrylic acid-methyl acrylate-methyl methacrylate copolymer, hydroxypropyl cellulose acetate succinate, polyvinyl acetate phthalate and shellac.

In another embodiment, a pharmaceutical composition comprises granules, wherein the granules comprising an active layer comprising Proton Pump Inhibitor, Crospovidone and Potassium hydroxide; an intermediate coating layer formed on active layer; and an enteric coating layer which is formed on the intermediate coating layer, wherein Potassium hydroxide is present in an amount less than 0.2 part by weight based on one part by weight of Proton Pump Inhibitor.

In another embodiment, a pharmaceutical composition comprises granules, wherein the granules comprising an active layer comprising Dexlansoprazole, Crospovidone and potassium hydroxide; an intermediate coating layer formed on active layer; and an enteric coating layer which is formed on the intermediate coating layer, wherein potassium hydroxide is present in an amount less than 0.2 part by weight based on one part by weight of Dexlansoprazole.

In another embodiment, a pharmaceutical composition is comprises granules, wherein the granules comprising an active layer comprising Proton Pump Inhibitor, Crospovidone and basic inorganic salt; an intermediate coating layer formed on active layer; and an enteric coating layer which is formed on the intermediate coating layer, wherein the Crospovidone is present in an amount less than 0.5 part by weight based on one part by weight of Proton Pump Inhibitor, wherein basic inorganic salt is present in an amount less than 0.2 part by weight based on one part by weight of Proton Pump Inhibitor.

In another embodiment, a pharmaceutical composition is comprises granules, wherein the granules comprising an active layer comprising Dexlansoprazole, Crospovidone and basic inorganic salt; an intermediate coating layer formed on active layer; and an enteric coating layer which is formed on the intermediate coating layer, wherein the Crospovidone is present in an amount less than 0.5 part by weight based on one part by weight of Dexlansoprazole, wherein basic inorganic salt is present in an amount less than 0.2 part by weight based on one part by weight of Dexlansoprazole.

In another embodiment, a pharmaceutical composition is capsule comprises granules, wherein the granules comprising an active layer comprising Dexlansoprazole, Crospovidone and potassium hydroxide; an intermediate coating layer formed on active layer; and an enteric coating layer which is formed on the intermediate coating layer, wherein the Crospovidone is present in an amount less than 0.5 part by weight based on one part by weight of Dexlansoprazole, wherein potassium hydroxide is present in an amount less than 0.2 part by weight based on one part by weight of Dexlansoprazole.

In another embodiment, a pharmaceutical composition comprises granules, wherein the granules comprising an active layer comprising proton pump inhibitor, Crospovidone and basic inorganic salt; an intermediate coating layer formed on active layer; and an enteric coating layer which is formed on the intermediate coating layer, wherein the basic inorganic salt is present in an amount less than 0.2 part by weight based on one part by weight of proton pump inhibitor, wherein the average particle size of the granules is about 400 micrometer or more, preferably average particle size of granules is about 500 micrometer to about 2500 micrometer.

The terms “average particle size”, “d₅₀” and “mass mean diameter” can be used interchangeably.

The average particle size, i.e. the average equivalent diameter, is defined as the diameter where 50 mass % of the particles of the Proton Pump Inhibitor have a larger equivalent diameter, and the other 50 mass-% have a smaller equivalent diameter.

The “average particle size” also refers to the median particle diameter based on mass (i.e. the particle diameter where one half of the mass of particles is contributed by particles with a lesser diameter and one half of the mass of particles is contributed by particles with a greater diameter).

The particle size can be measured using various commonly available methods such as measurement using light (eg. light-scattering methods or turbidimetric methods), sedimentation methods (eg. pipette analysis using an Andreassen pipette, sedimentation scales, photo-sedimentometers or sedimentation in a centrifugal force), pulse methods (eg. Coulter counter), sieving method or sorting by means of gravitational or centrifugal force.

When the particle size is smaller, it is difficult to increase concentration of the Proton Pump Inhibitor because a surface area becomes larger, which requires a larger amount of an enteric layer or agent. That is, in the present invention, it has been possible to decrease the amount of an enteric layer or agent by setting the particle size of at least about 400 micrometer or more, thereby increasing the concentration of the benzimidazole compound.

The granules of the present invention can be prepared by a known granulation method. Examples of the granulation method include a rotary granulation method (e.g. centrifugal Fluid-bed granulation method), a fluidized granulation method, an agitation granulation method (e.g. agitator fluidized granulation method) and the like. Among them, a rotary granulation method and an agitation granulation method (agitator fluidized granulation method) are preferred.

Specific examples of the rotary granulation method include CF apparatus manufactured by Freund, etc. Specific examples of the agitator fluidized granulation method include methods using Spiral Flow manufactured by Freund, Multiplex manufactured by Powlex, New Malume manufactured by Fuji Powdal and the like. A method for spraying a binder solution can be appropriately selected according to the kind of a granulator and, for example, it may be any of a top spraying manner, a bottom spraying manner, a tangential spraying manner and the like.

In another embodiment, a pharmaceutical composition comprises granules wherein the granules comprising an active layer comprising Dexlansoprazole, Crospovidone and basic inorganic salt; an intermediate coating layer formed on active layer; and an enteric coating layer which is formed on the intermediate coating layer, wherein the basic inorganic salt is present in an amount less than 0.2 part by weight based on one part by weight of Dexlansoprazole, wherein the average particle size of the granules is about 400 micrometer or more, preferably average particle size of granules is about 500 micrometer to about 2500 micrometer.

In another embodiment, a pharmaceutical composition comprises granules wherein the granules comprising an active layer comprising Dexlansoprazole, Crospovidone, Potassium hydroxide and one more pharmaceutically acceptable excipients; an intermediate coating layer formed on active layer; and an enteric coating layer which is formed on the intermediate coating layer, wherein Potassium hydroxide is present in an amount less than 0.2 part by weight based on one part by weight of Dexlansoprazole, wherein the average particle size of the granules is about 400 micrometer or more, preferably average particle size of granules is about 500 micrometer to about 2500 micrometer.

In another embodiment, a pharmaceutical composition is capsule comprises granules, wherein the granules comprising an active layer comprising Proton Pump Inhibitor, Crospovidone and basic inorganic salt; an intermediate coating layer formed on active layer; and an enteric coating layer which is formed on the intermediate coating layer, wherein the crospovidone is present in an amount less than 0.5 part by weight based on one part by weight of Proton Pump Inhibitor, wherein basic inorganic salt is present in an amount less than 0.2 part by weight based on one part by weight of Proton Pump Inhibitor, wherein the average particle size of the granules is about 400 micrometer or more, preferably average particle size of the granules is about 500 micrometer to about 2500 micrometer.

In another embodiment, a pharmaceutical composition is capsule comprises granules, wherein the granules comprising an active layer comprising Dexlansoprazole, Crospovidone and basic inorganic salt; an intermediate coating layer formed on active layer; and an enteric coating layer which is formed on the intermediate coating layer, wherein the crospovidone is present in an amount less than 0.5 part by weight based on one part by weight of Dexlansoprazole, wherein basic inorganic salt is present in an amount less than 0.2 part by weight based on one part by weight of Dexlansoprazole, wherein the average particle size of the granules is about 400 micrometer or more, preferably average particle size of granules is about 500 micrometer to about 2500 micrometer.

In another embodiment, a pharmaceutical composition is capsule comprises granules, wherein the granules comprising an active layer comprising Dexlansoprazole, Crospovidone and potassium hydroxide; an intermediate coating layer formed on active layer; and an enteric coating layer which is formed on the intermediate coating layer, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole, wherein the average particle size of the granules is about 400 micrometer or more, preferably average particle size of granules is about 500 micrometer to about 2500 micrometer.

In another embodiment, capsules can be made up of gelatin, hydroxypropylmethyl cellulose, pullulan, shellac or a combination thereof.

In another embodiment, a pharmaceutical composition is hard gelatin capsule comprises granules, wherein the granules comprising an active layer comprising Dexlansoprazole, Crospovidone and potassium hydroxide; an intermediate coating layer formed on active layer; and an enteric coating layer which is formed on the intermediate coating layer, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole, wherein the average particle size of the granules is about 400 micrometer or more, preferably average particle size of granules is about 500 micrometer to about 2500 micrometer.

The term ‘pharmaceutically acceptable excipient(s)’ used in pharmaceutical compositions of invention comprise but not limited to release controlling agents, diluents, binders, pH stabilizing agents, disintegrants, surfactants, glidants and lubricants.

The amounts of excipient(s) employed will depend upon how much active agent is to be used. One excipient(s) can perform more than one function.

The release controlling agents is hydrophilic, hydrophobic or combinations thereof.

The hydrophilic release controlling agent(s) according to invention comprises but not limited cellulose derivatives, alginic acid derivatives, polysaccharides, alkylene oxides or mixtures thereof. Preferably, hydrophilic release controlling agent(s) comprises celluloses or their salts or derivatives thereof, hydroxyethylcellulose, hydroxypropyl cellulose, hydroxypropyl methylcellulose (hypromellose), sodium carboxymethyl cellulose, alginic acid or their salts and derivatives thereof, carbomer (Carbopol(™)), polyethyleneoxide, xanthan gum, guar gum, locust bean gum, poly vinyl acetate, polyvinyl alcohol, lactose.

The hydrophobic release controlling agent(s) according to the invention comprises but not limited to hydrogenated vegetable oils, polymethacrylates, ethyl cellulose or mixtures thereof.

Preferably, hydrophobic release controlling agent(s) comprises Ammonio methacrylate copolymers type A and B as described in USP, methacrylic acid copolymer type A, B and C as described in USP, Polyacrylate dispersion 30% as described in Ph. Eur., Polyvinyl acetate dispersion, ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate). Poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), waxes such as beeswax, carnauba wax, microcrystalline wax, and ozokerite; fatty alcohols such as cetostearyl alcohol, stearyl alcohol; cetyl alcohol and myristyl alcohol; and fatty acid esters such as glyceryl monostearate, glycerol distearate; glycerol monooleate, acetylated monoglycerides, tristearin, tripalmitin, cetyl esters wax, glyceryl palmitostearate, glyceryl behenate, and hydrogenated castor oil.

Binders as used in the invention comprises but are not limited to, starches such as potato starch, wheat starch, corn starch; microcrystalline cellulose such as products known under the registered trademarks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents, as used in the invention comprises but not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants as used in the invention comprises but not limited to Mg, Al, Ca or Zn stearate, polyethylene glycol, glyceryl behenate, mineral oil, sodium stearyl fumarate, stearic acid, hydrogenated vegetable oil and talc.

Glidants comprises but not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

Disintegrants comprises but not limited to starches; clays; celluloses; alginates; gums; cross-linked polymers, cross-linked sodium carboxymethylcellulose or croscarmellose sodium, e.g., AC-DI-SOL from FMC; and cross-linked calcium carboxymethylcellulose; soy polysaccharides; and guar gum. Use of disintegrant according to the invention facilitates in the release of drug in the latter stage and thereby completely releasing the drug from the dosage form.

The pharmaceutical composition may optionally contain a surface-active agent. The preferred agent is copolymers composed of a central hydrophobic chain of polyoxypropylene (poly(propylene oxide)) and polyoxyethylene (poly(ethylene oxide)) that is well known as poloxamer. However, other agents may also be employed such as dioctyl sodium sulfosuccinate (DSS), triethanolamine, sodium lauryl sulphate (SLS), polyoxyethylene sorbitan and poloxalkol derivatives, quaternary ammonium salts or other pharmaceutically acceptable surface-active agents known to one ordinary skilled in the art.

The pharmaceutical composition can be formed by various processes known in the art but not limited to such as by dry granulation, wet granulation, melt granulation, direct compression, double compression, extrusion spheronization, layering and the like. The solvent(s) used in wet granulation include all the solvents well known in the art or the mixtures thereof.

In another embodiment, a stable pharmaceutical composition comprises granules, wherein the granules comprising an active layer comprising Dexlansoprazole, Crospovidone and potassium hydroxide; an intermediate coating layer formed on active layer; and an enteric coating layer which is formed on the intermediate coating layer, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole, wherein the composition comprises compound of formula (I)

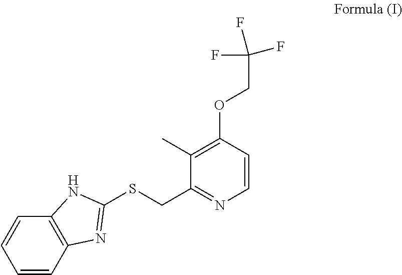 [SMILES: Cc1c(OCC(F)(F)F)ccnc1CSc1nc2ccccc2[nH]1]

in an amount less than about 0.3% when the composition is stored at 40° C. temperature and 75% RH (Relative Humidity) conditions for about 3 months.

In another embodiment, a stable pharmaceutical composition comprises granules, wherein the granules comprising an active layer comprising crystalline Dexlansoprazole, Crospovidone and potassium hydroxide; an intermediate coating layer formed on active layer; and an enteric coating layer which is formed on the intermediate coating layer, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole.

In another embodiment, a stable pharmaceutical composition comprises granules, wherein the granules comprising an active layer comprising amorphous Dexlansoprazole, Crospovidone and potassium hydroxide; an intermediate coating layer formed on active layer; and an enteric coating layer which is formed on the intermediate coating layer, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole.

In another embodiment, a pharmaceutical composition comprises granules, wherein the granules comprising an active layer comprising Proton Pump Inhibitor, Crospovidone and basic inorganic salt; an intermediate coating layer formed on active layer; and an enteric coating layer which is formed on the intermediate coating layer, wherein the crospovidone is present in an amount less than 0.5 part by weight based on one part by weight of Proton Pump Inhibitor, wherein basic inorganic salt is present in an amount less than 0.2 part by weight based on one part by weight of Proton Pump Inhibitor, wherein the Proton Pump Inhibitor is released in the pH range of no less than 5.0 to no more than 6.0.

In another embodiment, a pharmaceutical composition comprises granules, wherein the granules comprising an active layer comprising Dexlansoprazole, Crospovidone and basic inorganic salt; an intermediate coating layer formed on active layer; and an enteric coating layer which is formed on the intermediate coating layer, wherein the crospovidone is present in an amount less than 0.5 part by weight based on one part by weight of Dexlansoprazole, wherein basic inorganic salt is present in an amount less than 0.2 part by weight based on one part by weight of Dexlansoprazole, wherein the Dexlansoprazole is released in the pH range of no less than 5.0 to no more than 6.0.

In another embodiment, a pharmaceutical composition is capsule comprises granules, wherein the granules comprising an active layer comprising Dexlansoprazole, Crospovidone and potassium hydroxide; an intermediate coating layer formed on active layer; and an enteric coating layer which is formed on the intermediate coating layer, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole, wherein the Dexlansoprazole is released in the pH range of no less than 5.0 to no more than 6.0.

In another embodiment, a pharmaceutical composition comprising a proton pump inhibitor, crospovidone, basic inorganic salt and one or more pharmaceutically acceptable excipient, said composition having an in-vitro dissolution profile, when measured in 500 ml of 0.1 N HCl for 2 hours followed by 900 ml, pH 7.0 Phosphate Buffer with 5 mM Sodium Lauryl Sulphate as medium using Apparatus USP I (Basket), at 100 rpm speed as follows (a) less than about 15% of the proton pump inhibitor is released after 130 mins, b) more than about 25% of the proton pump inhibitor is released after 160 mins

In another embodiment, a pharmaceutical composition comprising Dexlansoprazole, crospovidone, potassium hydroxide and one or more pharmaceutically acceptable excipient, said composition having an in-vitro dissolution profile, when measured in 500 ml of 0.1 N HCl for 2 hours followed by 900 ml, pH 7.0 Phosphate Buffer with 5 mM Sodium Lauryl Sulphate as medium using Apparatus USP I (Basket), at 100 rpm speed as follows (a) less than about 15% of the Dexlansoprazole is released after 130 mins, (b) more than about 25% of the Dexlansoprazole is released after 160 mins.

In another embodiment, a pharmaceutical composition comprises granules, wherein the granules comprising an active layer comprising Proton Pump Inhibitor, Crospovidone and basic inorganic salt; an intermediate coating layer formed on active layer; and an enteric coating layer which is formed on the intermediate coating layer, wherein the crospovidone is present in an amount less than 0.5 part by weight based on one part by weight of Proton Pump Inhibitor, wherein basic inorganic salt is present in an amount less than 0.2 part by weight based on one part by weight of Proton Pump Inhibitor, said composition having an in-vitro dissolution profile, when measured in 500 ml of 0.1 N HCl for 2 hours followed by 900 ml, pH 7.0 Phosphate Buffer with 5 mM Sodium Lauryl Sulphate as medium using Apparatus USP I (Basket), at 100 rpm speed as follows (a) less than about 15% of the Proton Pump Inhibitor is released after 130 mins, (b) more than about 25% of the Proton Pump Inhibitor is released after 160 mins.

In another embodiment, a pharmaceutical composition is capsule comprises granules, wherein the granules comprising an active layer comprising Dexlansoprazole, Crospovidone and potassium hydroxide; an intermediate coating layer formed on active layer; and an enteric coating layer which is formed on the intermediate coating layer, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole, said composition having an in-vitro dissolution profile, when measured in 500 ml of 0.1 N HCl for 2 hours followed by 900 ml, pH 7.0 Phosphate Buffer with 5 mM Sodium Lauryl Sulphate as medium using Apparatus USP I (Basket), at 100 rpm speed as follows (a) less than about 15% of the Dexlansoprazole is released after 130 mins, (b) more than about 25% of the Dexlansoprazole is released after 160 mins.

In another embodiment, a pharmaceutical composition comprises: composition (i) comprising Proton Pump Inhibitor, one or more pharmaceutically acceptable excipients, and enteric coating layer which is soluble at the pH greater than 5, and composition (ii) comprising Proton Pump Inhibitor, one or more pharmaceutically acceptable excipients, such that the Proton Pump Inhibitor is released in the pH less than 5.0.

In another embodiment, a pharmaceutical composition comprises: composition (i) comprising Proton Pump Inhibitor, Crospovidone, basic inorganic salt wherein composition (i) is surrounded by enteric coating layer which is soluble at the pH greater than 5.0, and composition (ii) comprising Proton Pump Inhibitor and one or more pharmaceutically acceptable excipients, such that the Proton Pump Inhibitor is released in the pH less than 5.0.

In another embodiment, a pharmaceutical composition is multi-layered tablet comprises: composition (i) comprising Proton Pump Inhibitor, Crospovidone, basic inorganic salt wherein composition (i) is surrounded by enteric coating layer which is soluble at the pH greater than 5.0, and composition (ii) comprising Proton Pump Inhibitor and one or more pharmaceutically acceptable excipients, such that the Proton Pump Inhibitor is released in the pH less than 5.0, wherein composition (i) is coated by composition (ii).

In another embodiment, a pharmaceutical composition is capsule comprises: composition (i) comprising Proton Pump Inhibitor, Crospovidone, basic inorganic salt wherein composition (i) is surrounded by enteric coating layer which is soluble at the pH greater than 5.0, and composition (ii) comprising Proton Pump Inhibitor, one or more pharmaceutically acceptable excipients, such that the Proton Pump Inhibitor is released in the pH less than 5.0.

In another embodiment, a pharmaceutical composition is capsule comprises: composition (i) in the form of granule comprising Proton Pump Inhibitor, Crospovidone, basic inorganic salt wherein composition (i) is surrounded by enteric coating layer which is soluble at the pH greater than 5.0, and composition (ii) comprising Proton Pump Inhibitor, one or more pharmaceutically acceptable excipients, such that the Proton Pump Inhibitor is released in the pH less than 5.0.

In another embodiment, a pharmaceutical composition is capsule comprises: composition (i) in the form of granule comprising Proton Pump Inhibitor, Crospovidone, basic inorganic salt, wherein composition (i) is surrounded by enteric coating layer which is soluble at the pH greater than 5.0, wherein the crospovidone is present in an amount less than 0.5 part by weight based on one part by weight of Proton Pump Inhibitor and composition (ii) comprising Proton Pump Inhibitor, one or more pharmaceutically acceptable excipients, such that the Proton Pump Inhibitor is released in the pH less than 5.0.

In another embodiment, a pharmaceutical composition is capsule comprises: composition (i) in the form of granule comprising Proton Pump Inhibitor, Crospovidone, basic inorganic salt, wherein composition (i) is surrounded by enteric coating layer which is soluble at the pH greater than 5.0, wherein the crospovidone is present in an amount less than 0.5 part by weight based on one part by weight of Proton Pump Inhibitor, wherein the basic inorganic salt is present in an amount less than 0.2 part by weight based on one part by weight of Proton Pump Inhibitor and composition (ii) comprising Proton Pump Inhibitor, one or more pharmaceutically acceptable excipients, such that the Proton Pump Inhibitor is released in the pH less than 5.0.

In another embodiment, a pharmaceutical composition is capsule comprises: composition (i) in the form of granules comprising Dexlansoprazole, Crospovidone, basic inorganic salt, wherein composition (i) is surrounded by enteric coating layer which is soluble at the pH greater than 5.0, wherein the crospovidone is present in an amount less than 0.5 part by weight based on one part by weight of Dexlansoprazole, wherein the basic inorganic salt is present in an amount less than 0.2 part by weight based on one part by weight of Dexlansoprazole and composition (ii) comprising Dexlansoprazole, one or more pharmaceutically acceptable excipients, such that the Dexlansoprazole is released in the pH less than 5.0.

In another embodiment, a pharmaceutical composition is tablet comprises: composition (i) in the form of granule comprising Proton Pump Inhibitor, Crospovidone, basic inorganic salt wherein composition (i) is surrounded by enteric coating which is soluble at the pH greater than 5.0, and composition (ii) comprising Proton Pump Inhibitor, one or more pharmaceutically acceptable excipients, such that the Proton Pump Inhibitor is released in the pH less than 5.0.

Dexilant® is the brandname for the Dual delayed release capsules formulation of Dexlansoprazole for oral administration, 30 mg and 60 mg marketed by Takeda Pharmaceuticals America, Inc.

Dexilant® Capsule contains enteric-coated granules consisting of Dexlansoprazole sugar spheres, magnesium carbonate, sucrose, low-substituted hydroxypropyl cellulose, titanium dioxide, hydroxypropyl cellulose, hypromellose 2910, talc, methacrylic acid copolymers, polyethylene glycol 8000, triethyl citrate, polysorbate 80, and colloidal silicon dioxide. The components of the capsule shell include the following inactive ingredients: hypromellose, carrageenan and potassium chloride. Based on the capsule shell color, blue contains FD&C Blue No. 2 and Aluminum Lake; gray contains ferric oxide and Aluminum Lake; and both contain titanium dioxide.

In another embodiment, a pharmaceutical composition comprising a Dexlansoprazole, crospovidone, basic inorganic salt and one or more pharmaceutically acceptable excipient, wherein the crospovidone is present in an amount less than 0.5 part by weight based on one part by weight of Dexlansoprazole; the basic inorganic salt is present in an amount less than 0.2 part by weight based on one part by weight of Dexlansoprazole, wherein Cₘₐₓ and AUC of the composition are within the limit of 80% to 125% of Cₘₐₓ and AUC of the dual delayed release capsule formulation of Dexlansoprazole.

The term “Cₘₐₓ” as used herein, means maximum plasma concentration of Dexlansoprazole produced by the ingestion of the pharmaceutical composition of invention or the marketed Dexilant® (Dual delayed release capsules formulation of Dexlansoprazole) composition. Cₘₐₓ or peak plasma level may be used interchangeably.

The term “AUC” as used herein, means area under the plasma concentration-time curve of Dexlansoprazole produced by the ingestion of the pharmaceutical composition of invention or the marketed Dexilant® (Dual delayed release capsules formulation of Dexlansoprazole).

In another embodiment, a pharmaceutical composition comprising Dexlansoprazole, crospovidone, basic inorganic salt and one or more pharmaceutically acceptable excipient, wherein the crospovidone is present in an amount less than 0.5 part by weight based on one part by weight of Dexlansoprazole; the basic inorganic salt is present in an amount less than 0.2 part by weight based on one part by weight of Dexlansoprazole, wherein Cₘₐₓ and AUC of the pharmaceutical composition are independent of the intake of food.

In another embodiment, a pharmaceutical composition comprising Dexlansoprazole, crospovidone, basic inorganic salt and one or more pharmaceutically acceptable excipient, wherein the crospovidone is present in an amount less than 0.5 part by weight based on one part by weight of Dexlansoprazole; the basic inorganic salt is present in an amount less than 0.2 part by weight based on one part by weight of Dexlansoprazole, wherein the composition is bioequivalent to Dexilant® (Dual delayed release capsules formulation of Dexlansoprazole) in a bioavailability study in humans.

In another embodiment, a method of treating heartburn, acid reflux or gastroesophageal reflux disease in a patient, comprising administering a pharmaceutical composition comprising a Dexlansoprazole, crospovidone, basic inorganic salt and one or more pharmaceutically acceptable excipient, wherein the crospovidone is present in an amount less than 0.5 part by weight based on one part by weight of Dexlansoprazole; the basic inorganic salt is present in an amount less than 0.2 part by weight based on one part by weight of Dexlansoprazole.

In another embodiment, a method of treating heartburn, acid reflux or gastroesophageal reflux disease in a patient, comprising administering a pharmaceutical composition which is capsule comprises granules, wherein the granules comprising an active layer comprising Dexlansoprazole, Crospovidone and potassium hydroxide; an intermediate coating layer formed on active layer; and an enteric coating layer which is formed on the intermediate coating layer, wherein the crospovidone is present in an amount less than 0.5 part by weight based on one part by weight of Dexlansoprazole, wherein potassium hydroxide is present in an amount less than 0.2 part by weight based on one part by weight of Dexlansoprazole, wherein the Dexlansoprazole is released in the pH range of no less than 5.0 to no more than 6.0

In another embodiment, a pharmaceutical composition of invention can be used for the treatment and prevention of digestive ulcer (e.g., gastric ulcer, duodenal ulcer, stomal ulcer, Zollinger-Ellison syndrome, etc.), gastritis, reflux esophagitis, NUD (non-ulcer dyspepsia), gastric cancer and gastric MALT lymphoma; Helicobacter pylori eradication; suppression of upper gastrointestinal hemorrhage due to digestive ulcer, acute stress ulcer and hemorrhagic gastritis; suppression of upper gastrointestinal hemorrhage due to invasive stress (stress from major surgery necessitating intensive management after surgery, and from cerebral vascular disorder, head trauma, multiple organ failure and extensive burns necessitating intensive treatment); treatment and prevention of ulcer caused by a nonsteroidal anti-inflammatory agent; treatment and prevention of hyperacidity and ulcer due to postoperative stress; pre-anesthetic administration etc.

The following examples are illustrative of the present invention, and the examples should not be considered as limiting the scope of this invention in any way, as these examples and other equivalents thereof will become apparent to those versed in the art, in the light of the present disclosure, and the accompanying claims.

### EXAMPLES

### Example 1

| | | |
|---|---|---|
| Sr. No. | Ingredients | mg/capsule |

Stage-I: Seal Coating

| | | |
|---|---|---|
| 1 | Sugar Spheres | 56 |
| 2 | Hypromellose | 4 |
| 3 | Solvent** | qs |

| | |
|---|---|
| Total Weight | 60 |

Stage-II: Drug Loading

| | | |
|---|---|---|
| 4 | Dexlansoprazole | 60 |
| 5 | seal coated Sugar spheres | 60 |
| 6 | Potassium Hydroxide | 9 |
| 7 | Povidone | 25 |
| 8 | Crospovidone | 12 |
| 9 | Talc | 3 |
| 10 | Solvent ** | qs |

| | |
|---|---|
| Total Weight | 169 |

Stage-III: Barrier Coating

| | | |
|---|---|---|
| 11 | Drug Loaded Pellets | 169 |
| 12 | Hypromellose | 24 |
| 13 | Solvent ** | qs |

| | |
|---|---|
| Total Weight | 193 |

Stage-IV: Enteric coating

| | | |
|---|---|---|
| 14 | Barrier Coated Pellets | 193 |
| 15 | Enteric Polymer | 70 |
| 16 | Triethyl Citrate | 7 |
| 17 | Talc | 10 |
| 18 | Solvent ** | qs |

| | |
|---|---|
| Total Weight | 280 |

Stage-V Blending

| | | |
|---|---|---|
| 16 | Talc | 0.5 |
| 17 | Colloidal Silicone Dioxide | 0.5 |

| | |
|---|---|
| Total Weight of Pellets/Capsule | 281 |

**Evaporates during process

### Manufacturing Process:

1) Coat the solution of Hypromellose in a solvent on to sugar spheres.
2) Dissolve Potassium Hydroxide, Dexlansoprazole, Povidone, Crospovidone and Talc in a solvent to form dispersion and coat the dispersion on sugar spheres of step 1 and dry to form active layer.
3) Coat the solution of Hypromellose in a solvent on active layer and dry to form intermediate layer.
4) Coat the dispersion of Enteric Polymer, Triethyl Citrate and Talc in a solvent on intermediate coating layer of step 3 and dry to form enteric coating layer.
5) Sift the Talc and Colloidal Silicone Dioxide and blend with pellets of Step 4.
6) Fill enteric coated pellets of Step 5 in hard gelatine capsules.

### Example 2

| | | |
|---|---|---|
| Sr. No. | Ingredients | mg/capsule |

Stage-I: Seal Coating

| | | |
|---|---|---|
| 1 | Sugar Spheres | 56 |
| 2 | Hypromellose | 4 |
| 3 | Solvent ** | qs |

| | |
|---|---|
| Total Weight | 60 |

Stage-II: Drug Loading

| | | |
|---|---|---|
| 4 | Dexlansoprazole | 60 |
| 5 | seal coated Sugar spheres | 60 |
| 6 | Potassium Hydroxide | 9 |
| 7 | Povidone | 25 |
| 8 | Crospovidone | 12 |
| 9 | Talc | 3 |
| 10 | Solvent ** | qs |

| | |
|---|---|
| Total Weight | 169 |

Stage-III: Barrier Coating

| | | |
|---|---|---|
| 11 | Drug Loaded Pellets | 169 |
| 12 | Hypromellose | 24 |
| 13 | Solvent ** | qs |

| | |
|---|---|
| Total Weight | 193 |

Stage-IV: Enteric coating

| | | |
|---|---|---|
| 14 | Barrier Coated Pellets | 193 |
| 15 | Enteric Polymer | 62 |
| 16 | Triethyl Citrate | 6.2 |
| 17 | Hydrophobic Polymer | 1.24 |
| 18 | Talc | 8 |
| 19 | Solvent ** | qs |

| | |
|---|---|
| Total Weight | 270.44 |

Stage-V Blending

| | | |
|---|---|---|
| 20 | Talc | 0.5 |
| 21 | Colloidal Silicone Dioxide | 0.5 |

| | |
|---|---|
| Total Weight of Pellets/Capsule | 271.44 |

** Evaporates during process

### Manufacturing Process:

1) Coat the solution of Hypromellose in a solvent on to sugar spheres.
2) Dissolve Potassium Hydroxide, Dexlansoprazole, Povidone, Crospovidone and Talc in a solvent to form dispersion and coat the dispersion on sugar spheres of step 1 and dry to form active layer.
3) Coat the solution of Hypromellose in a solvent on active layer and dry to form intermediate layer.
4) Coat the dispersion of Enteric Polymer, Triethyl Citrate, Hydrophobic Polymer and Talc in a solvent on intermediate coating layer of step 3 and dry to form enteric coating layer.
5) Sift the Talc and Colloidal Silicone Dioxide and blend with pellets of Step 4.
6) Fill enteric coated pellets of step 5 in hard gelatine capsules.\

### Example 3

| | | |
|---|---|---|
| Sr. No. | Ingredients | mg/Capsule |

Stage-I: Seal Coating

| | | |
|---|---|---|
| 1 | Sugar Spheres | 56 |
| 2 | Hypromellose | 4 |
| 3 | Solvent** | qs |

| | |
|---|---|
| Total Weight | 60 |

Stage-II: Drug Loading

| | | |
|---|---|---|
| 4 | Dexlansoprazole | 57 |
| 5 | seal coated Sugar spheres | 60 |
| 6 | Potassium Hydroxide | 8.5 |
| 7 | Povidone | 23 |
| 8 | Crospovidone | 11.5 |
| 9 | Talc | 3 |
| 10 | Solvent** | qs |

| | |
|---|---|
| Total Weight | 163 |

Stage-III: Barrier Coating

| | | |
|---|---|---|
| 11 | Drug Loaded Pellets | 163 |
| 12 | Hypromellose | 24 |
| 13 | Solvent** | qs |

| | |
|---|---|
| Total Weight | 187 |

Stage-IV: Enteric coating

| | | |
|---|---|---|
| 14 | Barrier Coated Pellets | 187 |
| 15 | Enteric Polymer | 70 |
| 16 | Triethyl Citrate | 7 |
| 17 | Talc | 10 |
| 18 | Solvent** | qs |

| | |
|---|---|
| Total Weight | 274 |

Stage-III: Barrier Coating

| | | |
|---|---|---|
| 19 | Enteric Coated Pellets | 274 |
| 20 | Hypromellose | 27 |
| 21 | Solvent** | qs |

| | |
|---|---|
| Total Weight | 301 |

Stage-V: Second Drug Loading

| | | |
|---|---|---|
| 22 | Barrier coated Pellets | 301 |
| 23 | Dexlansoprazole | 3.0 |
| 24 | Potassium Hydroxide | 0.5 |
| 25 | Povidone | 1.25 |
| 26 | Crospovidone | 0.6 |
| 27 | Talc | 0.1 |
| 28 | Solvent** | qs |

| | |
|---|---|
| Total Weight | 306.45 |

Stage-V Blending

| | | |
|---|---|---|
| 29 | Talc | 0.5 |
| 30 | Colloidal Silicone Dioxide | 0.5 |

| | |
|---|---|
| Total Weight of Pellets/Capsule | 307.45 |

**Evaporates during process

### Manufacturing Process:

1) Coat the solution of Hypromellose in solvent on to sugar spheres.
2) Dissolve Potassium Hydroxide, Dexlansoprazole, Povidone, Crospovidone and Talc in a solvent to form dispersion and coat the dispersion on sugar spheres of step 1 and dry to form active layer.
3) Coat the solution of Hypromellose in a solvent on active layer and dry to form intermediate layer.
4) Coat the dispersion of Enteric Polymer, Triethyl Citrate and Talc in a solvent on intermediate coating layer of step 3 and dry to form enteric coating layer.
5) Coat the solution of Hypromellose in solvent on enteric coating layer and dry to form barrier coating layer.
6) Dissolve Potassium Hydroxide, Dexlansoprazole, Povidone, Crospovidone and Talc in a solvent to form dispersion and coat the dispersion on barrier coating layer of Step 5 and dry to form second active layer coated pellets.
7) Sift the Talc and Colloidal Silicone Dioxide and blend with pellets of Step 6.
8) Fill pellets of Step 7 in hard gelatine capsules.

### Example 4

| | | |
|---|---|---|
| Sr. No. | Ingredients | mg/capsule |

Wet Granulation

| | | |
|---|---|---|
| 1 | Dexlansoprazole | 60 |
| 2 | Mannitol | 120 |
| 3 | Crospovidone | 12 |
| 4 | Potassium Hydroxide | 9 |
| 5 | Povidone | 25 |
| 6 | Magnesium Stearate | 3 |
| 7 | Solvent** | qs |

| | |
|---|---|
| Total Tablet Weight | 229 |

Barrier coating

| | | |
|---|---|---|
| 8 | Core Tablets | 229 |
| 9 | Hypromellose | 32 |
| 10 | Solvent** | qs |

| | |
|---|---|
| Total Tablet Weight | 261 |

Enteric coating

| | | |
|---|---|---|
| 11 | Barrier Coated Tablets | 261 |
| 12 | Enteric Polymer | 60 |
| 13 | Triethyl Citrate | 5 |
| 14 | Talc | 13 |
| 15 | Solvent ** | qs |

| | |
|---|---|
| Total Weight | 339 |

**Evaporates during process

### Manufacturing Process:

1) Dissolve Potassium Hydroxide, Dexlansoprazole, Povidone, Crospovidone and Talc in a solvent.
2) Load Mannitol in Fluid Bed Processor and start fluidisation.
3) Top Spray the dispersion of Step 1 on to Step 2 using a Fluid Bed Processor and dry.
4) Size the dried blend of step 3 by passing through suitable mesh and add Magnesium stearate.
5) Compress the lubricated blend of Step 4 into tablets using suitable punch tooling on compression machine.
6) Coat the solution of Hypromellose in solvent on to step 5 in a coating pan to form barrier coating layer.
7) Dissolve Enteric Polymer, Talc and Triethyl Citrate in a solvent to form dispersion.
8) Spray the dispersion of step 7 on to step 6 in a coating pan to form enteric coated tablets.

### X-RD Stability Study:

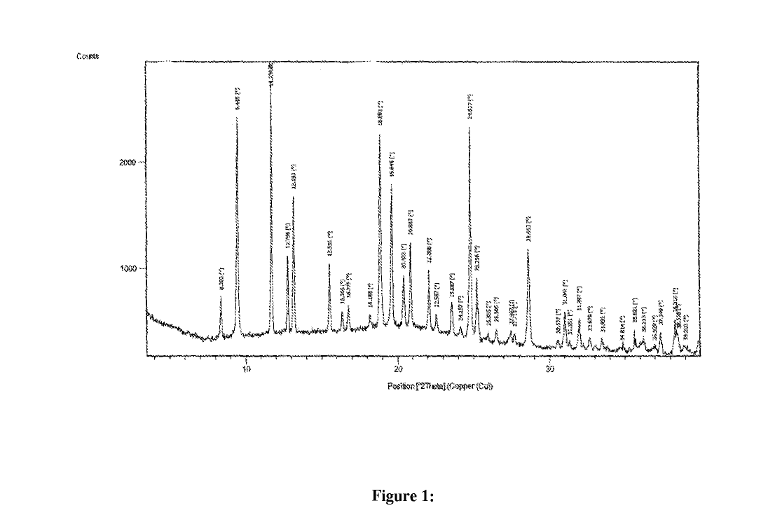

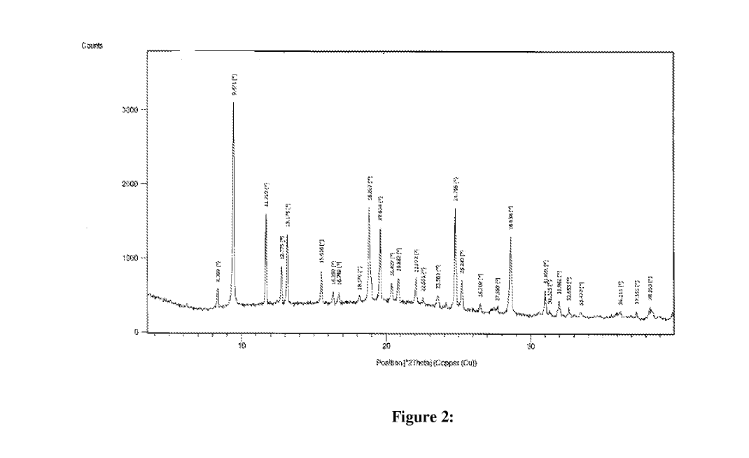

The pharmaceutical composition of Example 1 was kept for stability study at 40° C. temperature and 75% RH (Relative Humidity) conditions for 3 months. The pattern X-ray diffraction for the pharmaceutical composition of Example 1 obtained by measuring in X-ray diffractometer is shown in FIG. 1 (Initial) and FIG. 2 (after 3 months) and found to be stable.

### Stability Study of Dexlansoprazole Capsules:

Dexlansoprazole Capsules of Example 1 and 2 were stored in HDPE bottles under the condition of 40° C./75% RH for 3 months period and, thereafter, content of Dexlansoprazole was measured. The content was measured by HPLC assay method.

Based on the results disclosed in Table 1, it was revealed that the Dexlansoprazole capsules of Example 1 and 2 had stable content of Dexlansoprazole when stored under 40° C./75% RH for 3 months period.

TABLE 1

| | Example 1 | Example 2 |
|---|---|---|
| Duration | % Assay | % Assay |
| Initial | 101.2 | 104.2 |
| 1 Month | 101.5 | 104.6 |
| 2 Month | 101.6 | 103.5 |
| 3 Month | 102.0 | 103.2 |

### In-Vitro Dissolution Study:

Table 1 given below shows the dissolution profile of Dexlansoprazole Capsules of Example 1 and 2 of the present invention carried out in 500 ml of 0.1 N HCl for 2 hours followed by 900 ml, pH 7.0 Phosphate Buffer with 5 mM Sodium Lauryl Sulphate as medium using Apparatus USP I (Basket), at 100 rpm speed. The release profile (cumulative % of drug released) is given in Table 2.

TABLE 2

| | Cumulative % Drug Release | |
|---|---|---|
| | Dexlansoprazole | Dexlansoprazole |
| | Capsule, | Capsule, |
| Time in | 60 mg | 60 mg |
| minutes | Example 1 | Example 2 |
| 130 | 0.0 | 0.0 |
| 140 | 20.0 | 6.0 |
| 160 | 72.0 | 34.0 |
| 170 | 85.0 | 80.0 |
| 180 | 87.0 | 85.0 |
| 195 | 89.0 | 89.0 |
| 225 | 91.0 | 90.0 |
| 240 | 93.0 | 94.0 |

### In-Vivo Bioavailability Study:

An open label, balanced, randomized, single dose, three-treatment, three-period, crossover bioequivalence study of Reference product which is Dexilant® (Dual delayed release capsules formulation of Dexlansoprazole) 60 mg distributed by Takeda Pharmaceuticals America, Inc., Deerfield, Ill. 60015 and Test product which is Dexlansoprazole Capsule Composition of Example 1 of present invention administered under fed condition in healthy, adult, human male subjects under fed conditions. The study was designed to demonstrate the similar clinical efficacy compared to Dexilant® and the results as shown in the Table 3 below:

TABLE 3
Comparative pharmacokinetic parameters of
Example 1 vs Dexilant ® 60 mg in Fed state.

| | AUC (0-t) | AUC (0-∞) | Cₘₐₓ | |
|---|---|---|---|---|
| Product Name | ng · hr/ml | ng · hr/ml | ng/ml | Tₘₐₓ (hrs) |
| Dexlansoprazole | 17611.70 | 18999.43 | 2822.76 | 6.00 |
| Capsule 60 mg, | | | | |
| Example 1 | | | | |
| Dexilant ® Capsule, | 16636.62 | 18625.34 | 2567.39 | 7.00 |
| 60 mg | | | | |

1. A pharmaceutical composition comprising a proton pump inhibitor, crospovidone, basic inorganic salt and one or more pharmaceutically acceptable excipient, wherein
i. the crospovidone is present in an amount less than 0.5 part by weight based on one part by weight of the proton pump inhibitor;
ii. the basic inorganic salt is present in an amount less than 0.2 part by weight based on one part by weight of proton pump inhibitor.

2. The pharmaceutical composition according to claim 1, wherein the proton pump inhibitor selected from Lansoprazole, Omeprazole, Rabeprazole, Pantoprazole, Leminoprazole, Tenatoprazole, or an optically active isomer thereof, or a pharmaceutically acceptable salt thereof.

3. The pharmaceutical composition of claim 1, wherein the basic inorganic salt selected from basic salts of a sodium salt, a potassium salt, an aluminum salt, a magnesium salt or a calcium salt.

4. The pharmaceutical composition according to claim 1, wherein the proton pump inhibitor is Dexlansoprazole.

5. The pharmaceutical composition according to claim 4, wherein Cₘₐₓ and AUC of the composition are within the limit of 80% to 125% of Cₘₐₓ and AUC of the dual delayed release capsule formulation of Dexlansoprazole.

6. A pharmaceutical composition comprises granules, wherein the granules comprising an active layer comprising proton pump inhibitor, Crospovidone and basic inorganic salt, an intermediate coating layer formed on active layer, and an enteric coating layer formed on the intermediate coating layer, wherein the basic inorganic salt is present in an amount less than 0.2 part by weight based on one part by weight of proton pump inhibitor.

7. The pharmaceutical composition according to claim 6, wherein the average particle size of the granules is about 500 micrometer to 2500 micrometer.

8. The pharmaceutical composition according to claim 7, is a Capsule.

9. The pharamceutical composition according to claim 8, wherein proton pump inhibitor is Dexlansoprazole.

10. A method of treating heartburn, acid reflux or gastroesophageal reflux disease in a patient, comprising administering pharmceutical composition according to claim 9.
